(19) United States
(12) Patent Application Publication
(10) Pub. No.: US 2017/0028016 A1
(43) Pub. Date: Feb. 2, 2017

(54) METHODS OF TREATMENT OF C. DIFFICILE SPORES WITH RAMOPLANIN

(71) Applicant: Nanotherapeutics, Inc., Alachua, FL (US)

(72) Inventors: James D. Talton, Gainesville, FL (US); Carl N. Kraus, Raleigh, NC (US)

(21) Appl. No.: 15/304,179

(22) PCT Filed: Apr. 16, 2015
(86) PCT No.: PCT/US2015/026170

§ 371 Date: Oct. 14, 2016

Related U.S. Application Data
(60) Provisional application No. 61/980,370, filed on Apr. 16, 2014.

(51) Int. Cl. A61K 38/15; A61K 9/00

(52) U.S. Cl. CPC A61K 38/15; A61K 9/0053

(57) ABSTRACT

The present disclosure relates generally to methods of treatment of Clostridium difficile-associated disorders and/or C. difficile spores in the gastrointestinal tract by administering ramoplanin or a pharmaceutical formulation thereof, pharmaceutical compositions comprising ramoplanin, and therapeutic uses thereof in treating Clostridium difficile-associated disorders and/or C. difficile spores.

This application claims the benefit of U.S. Provisional Application No. 61/980,370, filed Apr. 16, 2014, which is incorporated herein by reference in its entirety.

### FIELD OF THE INVENTION

The present disclosure relates generally to methods of treatment of Clostridium difficile-associated conditions and/or Clostridium difficile (C. difficile) spores in the gastrointestinal tract by administering ramoplanin, oral formulations thereof, methods of preparing such compositions, and therapeutic uses thereof. The methods and oral compositions described herein allow ramoplanin to be administered by routes that are non-invasive to patients, such as by oral administration, for local delivery in the gastrointestinal tract of infected patients.

### BACKGROUND OF THE INVENTION

Ramoplanin is a glycolipodepsipeptide antibiotic that is bactericidal for many gram-positive aerobic and anaerobic bacteria, including C. difficile and has previously been evaluated in clinical development programs for both vancomycin-resistant enteroccocus (VRE) as well as the active treatment of C. difficile infection (CDI). Ramoplanin has been reported to also have activity against C. difficile spores, both in vitro and in an animal model, in, for example, U.S. Pat. No. 7,317,001 incorporated herein by reference. By “ramoplanin” is meant N/6686 or a preparation containing approximately 80% (with respect to the whole antibiotic substance, by HPLC assay) of A2 of A/16686 with a range of between 50-100%. The remaining portions consist essentially of small amounts of the related A and A′ factors of A/16686. Preparations of this type are currently obtained from pilot or semi-industrial fermentation and recovery operations described in detail in U.S. Pat. No. 4,303,646, incorporated herein by reference.

The natural history of CDI, regretfully, does not end with successful treatment of the infection, since a two to four week course of treatment with oral antibiotics, such as vancomycin, may only kill the vegetative cells and leave the spores intact. Approximately 25% of patients will experience a recurrence with higher rates among older age groups as well as increasing comorbidities. These relapses are thought to be at least partially due to residual spores in the human gut in clinically cured patients. Elimination of these spores could reduce relapse rates, decreasing the risk of readmission, further treatment requirements, or adverse clinical outcomes. An innovation gap exists for a compound that can successfully reduce such relapse rates as it reduce the risk of other nosocomial infections (e.g., VRE) and add a therapeutic regimen to the current armamentarium where currently none exists.

### SUMMARY

The present disclosure relates generally to methods of treatment of C. difficile-associated disorders and/or C. difficile spores in the gastrointestinal tract by administering ramoplanin or a pharmaceutical formulation thereof, pharmaceutical compositions comprising ramoplanin, and therapeutic uses thereof in treating C. difficile-associated disorders and/or C. difficile spores.

Thus, according to the present disclosure in one aspect, there is provided a method of treating a patient with a C. difficile-associated condition, said method comprising administering to said patient ramoplanin in an amount and for a duration effective to treat said condition, wherein said method is effective for reducing the population of C. difficile and rendering C. difficile spores non-viable, wherein the exosporium is the target for ramoplanin binding.

In some embodiments, the disclosure provides a method of rendering C. difficile spores non-viable, the method comprising administering ramoplanin to a patient in need thereof.

According to the present disclosure in a further aspect, there is provided a method of preventing a relapse of a C. difficile-associated disorder in a patient in need thereof, the method comprising administering ramoplanin to the patient.

In some embodiments, the patient is at risk for relapse of a C. difficile-associated condition.

In some embodiments, the patient was treated with at least one antimicrobial prior to administration of ramoplanin.

In some embodiments, the at least one antimicrobial is ineffective for eradicating or eliminating C. difficile spores, or rendering C. difficile spores non-viable.

In some embodiments, the at least one antimicrobial did not eradicate or eliminate C. difficile spores in the patient, or render C. difficile spores non-viable in the patient.

In some embodiments, the patient is at risk for further infection,

In some embodiments, the method limits resistance to at least one antimicrobial by limiting residual C. difficile population density.

The present disclosure is also directed to a method of treating a patient with a C. difficile-associated condition, wherein the method alleviates CDI institutional outbreaks through targeted infection control prophylaxis by analyzing a patients stool to test for C. difficile bacteria or spores and providing targeted prophylaxis of an identified carrier.

In some embodiments, the ramoplanin is administered as a pharmaceutical composition comprising ramoplanin.

In some embodiments, the ramoplanin or pharmaceutical composition thereof is administered orally.

### BRIEF DESCRIPTION THE DRAWINGS

FIG. 1. Spores incubated in water over the course of six days showed no significant variation in counts (p=0.24).

FIG. 2 Continuous exposure of spores in water to metronidazole, vancomycin, or ramoplanin. Only ramoplanin-exposed spores yielded no counts (p<0.001 for all time points).

FIG. 3. Unbound ramoplanin was recovered from spores exposed to two concentrations of ramoplanin for thirty minutes (300 and 600 μg/mL). Concentrations were stable from Day 1 (D1)-Day 28 (D28).

FIG. 4. Summary spore counts for all time points at day 28 after 30 minute ramoplanin exposure. Higher concentrations (300 and 600 μg/mL) resulted in no counts for the duration of the incubation in water.

FIG. 5. Diluting ramoplanin-exposed samples 10× resulted in growth not measurable at previous dilution.

FIG. 6. Removal of exosporium after exposure to ramoplanin resulted in counts equivalent to water exposure; unprocessed spores, similarly exposed resulted in no measurable counts.

### DESCRIPTION

The instant disclosure includes in vitro studies demonstrating an effect of Ramoplanin on C. difficile spores. Without wishing to be bound by any particular theory, the effect seems to not be sporicidal per se. Instead, it appears that the spore exosporium may be the target for ramoplanin binding, rendering the C. difficile spore non-viable, and/or permitting an “ambush” type of vegetative cell killing, initiating cell killing from residual exosporium-bound ramoplanin once the spore vegetates. It appears that ramoplanin, based on its relatively novel mechanism of action, non-absorbable kinetics (enhanced by dosing to non-inflamed gut mucosa) and established safety profile from prior studies would fill the above-mentioned treatment gap.

Accordingly, an objective of the instant disclosure is to provide a method of treating a C. difficile-associated condition in a patient in need thereof, the method comprising administering to the patient ramoplanin in an amount and for a duration effective to treat said condition. In some embodiments, the method can effective for reducing the population of C. difficile. In certain embodiments, the method can be effective at rendering C. difficile spores non-viable.

Another objective of the instant disclosure is to provide a method of rendering C. difficile spores non-viable by administering ramoplanin to a patient.

An object of the instant disclosure also includes providing a method of rendering C. difficile spores non-viable, the method comprising administering ramoplanin to a patient in need thereof.

A further objective of the present disclosure is to provide a method of preventing a relapse of a C. difficile-associated disorder in a patient in need thereof, the method comprising administering ramoplanin to the patient.

An additional objective of the instant disclosure is directed to a method of treating a patient with a C. difficile-associated condition to alleviate CDI institutional outbreaks through targeted infection control prophylaxis. In some embodiments, the method comprises analyzing a patients stool to test for C. difficile bacteria or spores and providing targeted prophylaxis of an identified carrier.

In some embodiments of the instant disclosure, the patient is at risk of relapse of a C. difficile-associated condition. In other embodiments of the instant disclosure, the patient was treated for a C. difficile-associated condition or was treated with antimicrobials prior to the administration of ramoplanin. In certain embodiments of the instant disclosure, the treatment for a C. difficile or antimicrobial treatment may not have eradicated or eliminated C. difficile spores from the patient. In some embodiments of the instant disclosure, the patient, for example, about 20% of hospitalized patients and nursing home residents, is at risk for further infection. In further embodiments of the instant disclosure, the methods limit the development of antimicrobial resistance by limiting residual C. difficile population densities.

### EXAMPLES

The following in vitro experiments have been conducted to consider the mechanism of action that ramoplanin may have on C. difficile spores.

### Example 1

### Dose/Dependent Sporadical Assays

C. difficile exposure at three levels of ramoplanin (2, 300, 600 μg/mL) and to water was evaluated. Three 1 mL aliquots from each exposure level (including the water control) were washed 6 times with distilled and deionized water (1 mL)—a process that does not detach C. difficile spore exosporia—before re-suspension in water (1 mL). The wash supernatant fluids (n=6/level) and the final spore suspensions (n=1/level) were counted by plating three 10-fold serial dilutions on NaTaurocholate Blood Agar (NaTBA). Each set of three counts were averaged and compared by t-test.

Ramoplanin-exposed C. difficile spores (ribotype 027), when plated, were non-viable at below fecal level concentrations of Ramoplanin (300 μg/mL). As used herein, “non-viable” means that the spores are unable to vegetate to C. difficile. The observed bactericidal activity of Ramoplanin was rapid with no apparent difference in effect vs. time (30 min of exposure results in maximal effect). The observed bactericidal activity does not appear to be related to antimicrobial carry-over as the effect persists after multiple washes (×6). However, there was a small observed rise in counts after the washes, which could be the result of leeching of ramoplanin from the exosporium in a concentration-dependent manner.

### Example 2

### Ramoplanin Recovery from Exosporium via Bioassay: Targeting the Exosporium

C. difficile (027) exposure (30 min/37 C) to multiple doses of ramoplanin and to water was evaluated. Following ramoplanin exposure for 30 minutes, sampling occurred at days D0, D1, D7, D14, and D28. Without wishing to be bound by any particular theory, sampling over time may characterize the duration of effect having clinical implication on the duration of oral therapy. From each level at each time point, three aliquots were taken (i.e. n=3 for each subsequent assay) and each washed 6 times before the spore re-suspension. Each wash (100 only) and spore suspension (100, 10-1 10-2) were counted on NaTBA and assayed for ramoplanin by a zone of inhibition assay using S. salivarius.

Spores exposed to 600 μg/mL ramoplanin with intact exosporium resulted in non-recoverable C. difficile. Spores exposed to 600 μg/mL ramoplanin then stripped of the exosporium resulted in high organism recovery.

### Example 3

### Longevity Assays

As above, C. difficile (027) was exposed to multiple doses of Ramoplanin (30 min/37 C) and to water, stored at 37 C (anaerobic), and sampled at days D0, D1 D7, D14, and D28. Without wishing to be bound by any particular theory, sampling over time may demonstrate that the ramoplanin effect is prolonged while any decline over time may imply a longer duration of oral therapy is required. From each level at each time, three aliquots were taken (i.e. n=3 for each subsequent assay) and each washed 6 times before the spore resuspension. Each wash (100 only) and spore suspension (100, 10-1 10-2) were counted on NaTBA.

There was no difference in recovery (no organisms are recoverable) from 30 min after ramoplanin exposure and wash through day 28. There was a decline in the bioassay's zone of inhibition over time which may imply leeching of ramoplanin from exposporium, but insufficient to permit vegetative cell survival on plating.

### Example 4

### Dependency of Bactericidal Activity on Exosporium Presence

From this example, it appears that the absence of a spore exosporium diminishes the bactericidal effect of Ramoplanin. Following enzyme digest and sonication of spore prep (Cortes et. al.) to removal the exosporium, the spores were exposed (027) to single dose of Ramoplanin (600 mcg/mL) vs water for 30 min and sampled (supernatant and prep) at 1 time point (T0).

Spores exposed to 600 μg/mL ramoplanin with intact exosporium resulted in non-recoverable C. difficile. Spores exposed to 600 μg/mL ramoplanin and stripped of the exosporium resulted in high organism recovery.

Without wishing to be bound by any particular theory, the growth-inhibitory levels of ramoplanin remained on ramoplanin-exposed spores after multiple washes. This effect was not present when spore exosporia were removed either before exposure or after ramoplanin exposure. Without wishing to be bound by any particular theory, the exosporium appears to be the target for ramoplanin binding to render C. difficile spores non-viable, and/or permitting an “ambush” type of vegetative cell killing once the spores are plated. Without wishing to be bound by any particular theory, direct sporicidal activity seems unlikely given the absence of an effect of ramoplanin on spores that had undergone exosporium processing.

The rapid and persistent activity of ramoplanin on C. difficile spores could have beneficial clinical effects in patients with recurrent C. difficile secondary to relapse.

Examples 5-7 below were conducted using the following bacterial strain and spore preparation: C. difficile spores (ribotype 027) were prepared as described in Wilson et al. 1982, “Use of sodium taurocholate to enhance spore recovery on a medium selective for C. difficile,” Journal of Clinical Microbiology 15:443-446. Spore pellets were resuspended in 1/10 the original volume in deionized water. The spores were counted by serial 10-fold dilution in PRAS dilution blanks (Anaerobe Systems, Morgan Hill, Calif.) and subsequently plated on BHI agar (anaerobic incubation for 48 hrs at 37±2° C.) supplemented with horse blood and sodium taurocholate, a known germinant. Id.

### Example 6

### Antimicrobial Survey

10⁵ spores/mL were stored in deionized water anaerobically at 37±2° C. for six days and continuously exposed to fecal-level concentrations of metronidazole (10 ug/mL), vancomycin (500 μg/mL), or ramoplanin (300 μg/mL). One mL of spores was sampled daily from Day 0-6 (D0 to D6) and serially diluted 10-fold, as above. Neither metronidazole nor vancomycin demonstrated spore-specific activity; hence such activity was assessed only for ramoplanin, using the same ribotype, 027. 10⁵ spores/mL were exposed to 0, 2, 300, or 600 μg/mL of ramoplanin for 30 minutes at 37±2° C. After 30 minutes, excess ramoplanin was removed by six sequential centrifugation steps, each requiring 10 minutes at 15,000×g with subsequent resuspension in deionized water. After the last wash, spores were reconstituted to the original volume with deionized water and stored at 37±2° C. for 28 days. At each time point (D0, 1, 7, 14, and 28), samples were centrifuged at 10,000×g for two minutes to separate spores from unbound ramoplanin. The spores were reconstituted to original sampling volume, diluted and plated as described above. Supernatants were held for the ramoplanin bioassay in EXAMPLE 6.

### Example 6

### Ramoplanin Bioassay

The procedures used for the bioassay followed those described by Carman et al. 2005. “Antibiotics in the human food chain: establishing no effect levels of tetracycline, neomycin, and erythromycin using a chemostat model of the human colonic microflora.” Regulatory Toxicology and Pharmacology, 43:168-180. Briefly, lawns of 10⁶ CFU/mL Streptococcus salivarius were plated on unsupplemented blood agar plates. 30 μL of each wash supernatant (day 0, 1, 7, 14, 28) were pipetted aseptically onto blank sterile paper discs which were transferred to the lawn of S. salivarius, in triplicate. Plates were incubated aerobically for 24 hours at 37±2° C. Zones of inhibition around each disc were measured using calipers and compared to standard curves of ramoplanin for which known concentrations of ramoplanin and measured zones of inhibition had been compared.

### Example 7

### Exosporia Processing

Spore exosporia were removed as described by Escobar-Cortes et al. 2013. Proteases and sonication specifically remove the exosporium layer of spores of C. difficile strain 630. Journal of microbiological methods, 93:25-31; “intact” spores did not undergo such processing. Briefly, spores were washed four times by centrifugation (10 minutes at 10,000×g) and resuspended in deionized water followed by final resuspension in 15 mL of PBS and sonicated for 90 seconds. Three mL of 10% Sarkosyl (detergent) were added to each preparation and subsequently incubated 15 minutes at room temperature with rocking. Preparations were then centrifuged for 10 minutes at 10,000×g, and pellets were resuspended in 10 mL PBS with 0.1 mL of 1M Tris and 10 mg of lysozyme. Spores were rocked overnight at 37±2° C. and then sonicated for 90 seconds, passed through a 50% solution of sucrose using a swinging bucket rotor for 20 minutes at 4,000×g, and resuspended in a solution containing 3 mL PBS, 200 mM EDTA, 300 ng/mL Proteinase K and 1% Sarkosyl. The spores were further rocked at room temperature for 20 minutes and were passed through a 50% solution of sucrose as previously described by Sabja et al, Id. Next, the spores were washed in deionized water by two centrifugation steps for 10 minutes at 10,000×g and resuspended in deionized water; plating and incubation were carried out as described in the foregoing text.

For comparisons among different samples, Microsoft Excel (2010) was used for statistical evaluation of one-way ANOVA (single-factor) as well as post-hoc testing (two-sample, assuming unequal variances).

### Results

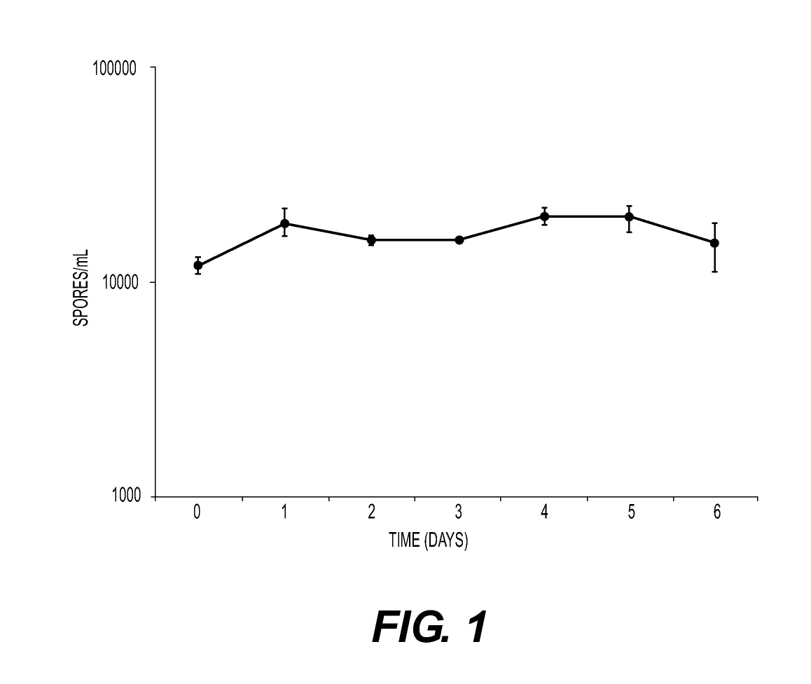

Spore stability in water: Incubation in water at 37° C. for six days resulted in spore persistence with limited variation in spore counts from D0 through D6 (FIG. 1). Mean spore counts (spores/mL) on D0 and D6 were 1.3×10⁴(±0.11×10⁴) and 1.1×10⁴(±0.39×10⁴), respectively, with no significant difference noted (p=0.240). Stable spore counts permitted assessment of continuous antimicrobial exposure in water with the expectation that no effect on spores would be identified, a finding consistent with previous observations. See Baines S D et al. 2009 “Activity of vancomycin against epidemic C. difficile strains in a human gut model,” The Journal of Antimicrobial Chemotherapy 63:520-525.

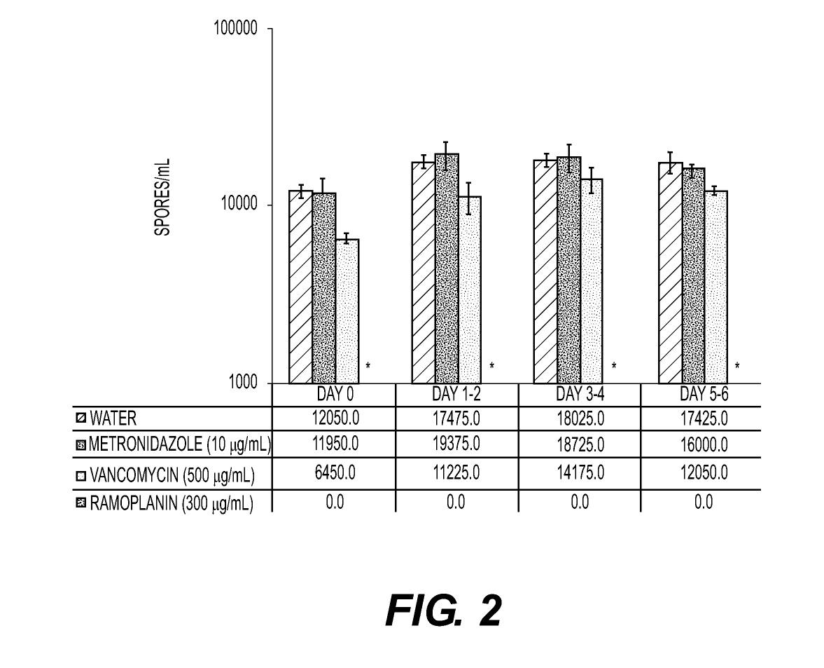

Antimicrobial activity on spores: The effects of the assessed antimicrobials on spores were distinct, with ramoplanin clearly having a more pronounced effect than vancomycin, metronidazole, or water controls. Continuous exposure to vancomycin (500 μg/mL), metronidazole (10 μg/mL) and water did not significantly reduce spore counts as compared to baseline. The spores exposed to ramoplanin (300 μg/mL) showed no growth after plating (FIG. 2), resulting in a significant decline (denoted by *) of spore counts over time based on ANOVA at each time point after baseline (D1-2, p=0.0002; D3-4, p=0.0001; D5-6, p<0.0001). Post-hoc, two-sample t-tests confirmed significant differences in spore counts between ramoplanin and comparators (water, metronidazole, and vancomycin) at the evaluated time points (ail p values <0.02). Not only were the differences in spore counts significant between ramoplanin and each comparator, but the magnitude of the effect was striking in that ramoplanin-exposed spores, when plated on agar, resulted in no observable growth. There was no statistical evidence of spore growth suppression by vancomycin or metronidazole when compared to baseline (as assessed by ANOVA; p=0.18 and p=0.49 respectively).

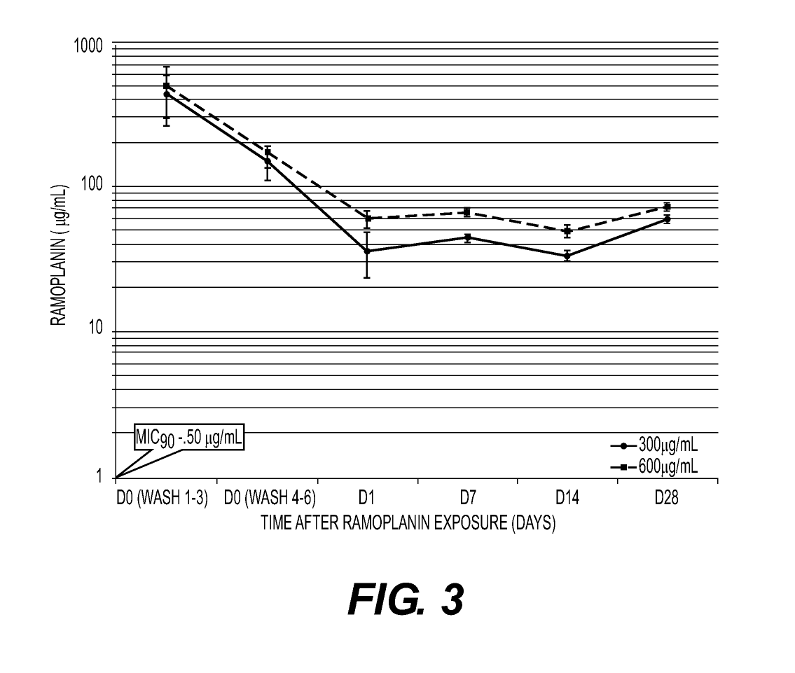

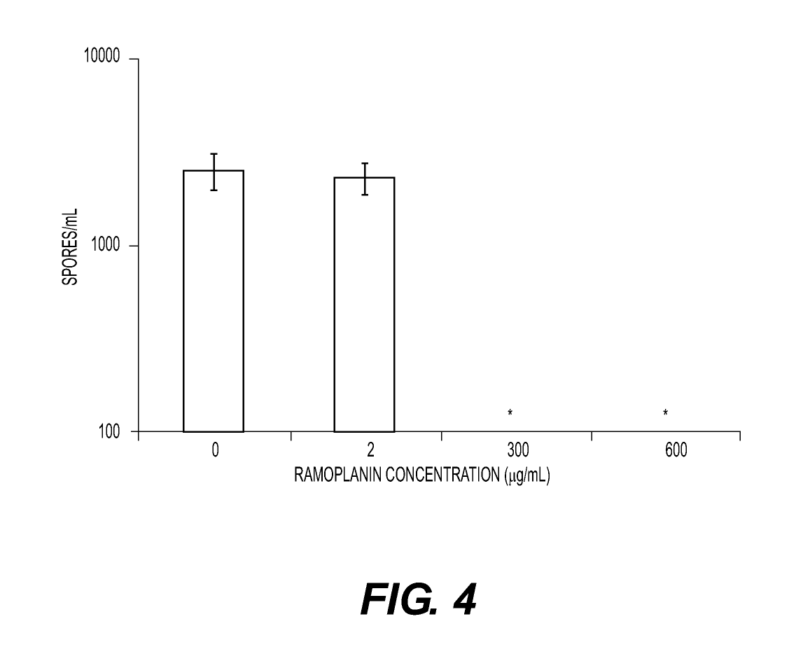

Persistence of ramoplanin activity: The observed effect of ramoplanin on spores was prolonged. Ramoplanin concentrations were varied (0 μg/mL, 2 μg/mL, 300 μg/mL, and 600 μg/mL) to characterize a concentration/response relationship for the drug's effects. Exposure to ramoplanin exposure was brief and limited to 30 minutes. Ramoplanin-exposed spores were washed six times on D0 to remove unbound drug and re-pelleted once at each time point. At set intervals (D0, D1, D7, D14 and D28), spore-bound and free ramoplanin concentrations were assayed (FIG. 3), and spores were counted (FIG. 4, no growth is denoted by *). From D1 through D28 supernatant concentrations of ramoplanin remained stable, ranging from (mean±SEM) 33.2±1.1 to 57.8±3.2 μg/mL for the 300 μg/mL-exposed spores and from 49.0±5.1 to 72.3±4.1 for the 600 μg/mL-exposed spores (FIG. 3). There were no significant differences in supernatant ramopianin concentrations among the evaluated time points for spores exposed to 300 μg/mL (p=0.116) or to 600 μg/mL (p=0.065).

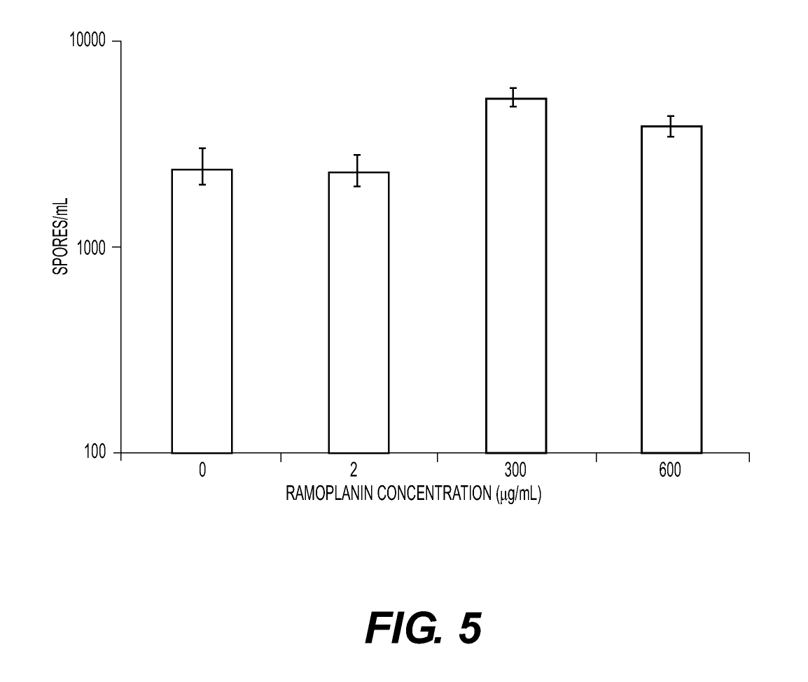

At each time point for which ramoplanin drug concentrations were assessed, spore pellets free of unbound ramoplanin were plated for spore counts to determine if the spore-related activity would persist past the short-term time points initially evaluated (D0-D6 of antimicrobial survey). Specifically, the intent was to determine whether a brief 30-minute ramoplanin exposure that was followed by multiple washes could inhibit spore growth over the course of 28 days. Those spores exposed to low concentrations of ramoplanin (2 μg/mL) demonstrated no inhibition relative to controls (0 μg/mL ramoplanin) over the course of 28 days (FIG. 4), with mean counts ±SEM of 2.3×10⁴±4.6×10² and 2.5×10⁴±5.71×10², respectively. Those spores exposed to higher concentrations of ramoplanin (300 μg/mL and 600 μg/mL) had no growth observed when plated undiluted. The difference in spore counts after ramoplanin exposure when compared to deionized water controls was significant (p<0.0001). However, diluting these samples 10× resulted in spore counts similar to the 0 and 2 μg/mL exposed spores (FIG. 5). At an additional dilution (10×), spore counts of samples exposed to higher concentrations of ramoplanin (300 μg/mL and 600 μg/mL), did not demonstrate a similar effect as was seen with the lower dilution spore counts relative to water controls (p=0.19 and p=0.53, respectively, paired two sample t-Test for means).

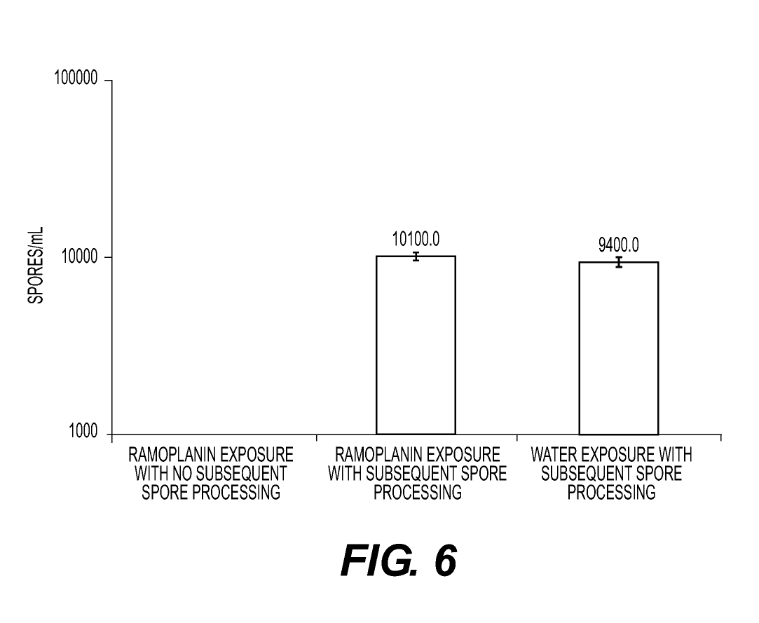

Adherence of ramoplanin to C. difficile exosporium: In order to determine whether the exosporium was essential to the observed growth-inhibiting effects of ramoplanin, the exosporia were processed as described in Escobar-Cortes et al, 2013, resulting in spores with “stripped” exosporia, and then compared to unprocessed spores (with “intact” exosporia) under three separate conditions (all ramoplanin concentrations were 300 μg/mL): (1) ramoplanin exposure with no subsequent spore processing (i.e., “intact”), (2) ramoplanin exposure with subsequent spore processing (i.e., “stripped”), and (3) water exposure with subsequent spore processing (FIG. 6). Ramoplanin exposed spores that had not been processed yielded no growth when plated; in contrast, when ramoplanin-exposed spores were processed (i.e., their exosporia had been removed), spore counts were similar to control, water-exposed spores. After ramoplanin exposure, the difference in spore counts between samples that were not processed (with presumed intact exposporia) and samples that were processed (without presumed exposporia) was significant (p<0.0001). No difference in spore count was noted when comparing processed spores that had been exposed to ramoplanin to spores exposed only to water (p=0.389).

Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

1. A method of treating a patient with a C. difficile-associated condition, said method comprising administering to said patient ramoplanin in an amount and for a duration effective to treat said condition, wherein said method is effective for reducing the population of C. difficile and rendering C. difficile spores non-viable, wherein the exosporium is the target for ramoplanin binding.

2. A method of rendering C. difficile spores non-viable, the method comprising administering ramoplanin to a patient in need thereof.

3. A method of preventing a relapse of a C. difficile-associated disorder in a patient in need thereof, the method comprising administering ramoplanin to the patient.

4. The method according to claim 1, wherein the patient is at risk for relapse of a C. difficile-associated condition.

5. The method according to claim 1, wherein the patient was treated with at least one antimicrobial prior to administration of ramoplanin.

6. The method according to claim 5, wherein the at least one antimicrobial is ineffective for eradicating or eliminating C. difficile spores, or rendering C. difficile spores non-viable.

7. The method according to claim 5, wherein the at least one antimicrobial did not eradicate or eliminate C. difficile spores in the patient, or render C. difficile spores non-viable in the patient.

8. The method according to claim 1, wherein the patient is at risk for further infection.

9. The method according to claim 1, wherein the method limits resistance to at least one antimicrobial by limiting residual C. difficile population density.

10. A method of treating a patient with a C. difficile-associated condition according to claim 1, wherein the method alleviates CDI institutional outbreaks through targeted infection control prophylaxis by analyzing a patient's stool to test for C. difficile bacteria or spores and providing targeted prophylaxis of an identified carrier.

11. The method according to claim 1, wherein the ramoplanin is administered as a pharmaceutical composition comprising ramoplanin.

12. The method according to claim 1, wherein the ramoplanin or pharmaceutical composition thereof is administered orally.

13. The method according to claim 2, wherein the patient is at risk for relapse of a C. difficile-associated condition, or was treated with at least one antimicrobial prior to administration of ramoplanin.

14. The method according to claim 13, wherein the at least one antimicrobial is ineffective for eradicating or eliminating C. difficile spores, or rendering C. difficile spores non-viable, or the at least one antimicrobial did not eradicate or eliminate C. difficile spores in the patient, or render C. difficile spores non-viable in the patient.

15. The method according to claim 2, wherein the ramoplanin is administered as a pharmaceutical composition comprising ramoplanin.

16. The method according to claim 2, wherein the ramoplanin or pharmaceutical composition thereof is administered orally.

17. The method according to claim 3, wherein the patient is at risk for relapse of a C. difficile-associated condition, or was treated with at least one antimicrobial prior to administration of ramoplanin.

18. The method according to claim 18, wherein the at least one antimicrobial is ineffective for eradicating or eliminating C. difficile spores, or rendering C. difficile spores non-viable, or the at least one antimicrobial did not eradicate or eliminate C. difficile spores in the patient, or render C. difficile spores non-viable in the patient.

19. The method according to claim 3, wherein the ramoplanin is administered as a pharmaceutical composition comprising ramoplanin.

20. The method according to claim 3, wherein the ramoplanin or pharmaceutical composition thereof is administered orally.
